(19) United States
(12) Patent Application Publication
(10) Pub. No.: US 2025/0019330 A1
(43) Pub. Date: Jan. 16, 2025

(54) A PROCESS AND APPARATUS FOR PRODUCING METHANOL

(71) Applicant: ANDRITZ OY, Helsinki (FI)

(72) Inventors: Otto GREIS, Helsinki (FI); Naveen CHENNA, Helsinki (FI); Aino PESOLA, Helsinki (FI); Pekka TERVOLA, Helsinki (FI)

(21) Appl. No.: 18/711,031

(22) PCT Filed: Nov. 16, 2022
(86) PCT No.: PCT/FI2022/050753

§ 371 Date: May 16, 2024

(30) Foreign Application Priority Data
Nov. 18, 2021 (FI) 20216182

(51) Int. Cl. C07C 29/50

(52) U.S. Cl. CPC C07C 29/50

(57) ABSTRACT

A process, and a system configured to perform the process, is provided which oxidizes lignin present in filtrates into methanol by feeding into a lignin-containing feedstock an oxidative agent in a reactor. The process includes: providing, in a reactor, a feedstock comprising lignin in an aqueous medium; feeding into the feedstock at least one oxidative agent to produce a reaction mixture in which methoxyl groups of the lignin are oxidized into methanol; adjusting the pH of the reaction mixture to a value in a range of 5 to 14; and operating the reactor at a temperature in a range of 50° C. to 100° C.

### TECHNICAL FIELD

The present disclosure generally relates to production of methanol. The disclosure relates particularly, though not exclusively, to a process and apparatus for producing methanol from fiber line filtrates that contain lignin.

### BACKGROUND

This section illustrates useful background information without admission of any technique described herein representative of the state of the art.

Fiber line filtrates of pulp mills contain various amounts of lignin. Previously, lignin has been used at pulp mills to produce heat and steam. However, there is a need to develop processes that use lignin to produce products with a higher value.

### SUMMARY

The appended claims define the scope of protection. Any example and/or technical description of an apparatus, system, product and/or process in the description and/or drawing which is not covered by the claims, is presented herein not as an embodiment of the invention but as background art or example useful for understanding the invention.

It is an object of the present disclosure to provide a process for producing methanol from lignin present in waste streams and/or side streams originating from industrial processes, such as from fiber line filtrate streams. Another object is to provide an alternative solution to existing technology in methanol production and/or in lignin processing.

According to a first aspect is provided a process for producing methanol comprising:
providing, in a reactor, a feedstock comprising lignin in an aqueous medium; and
feeding into the feedstock at least one oxidative agent to produce a reaction mixture wherein methoxyl groups of the lignin are oxidized into methanol.

When the oxidative agent(s) is fed into the feedstock, a reaction mixture is formed wherein at least one oxidative agent converts methoxyl groups present in lignin into methanol in oxidative reactions. In an embodiment both phenolic and non-phenolic lignin is oxidized by the present process.

In an embodiment the reactor is in fluid connection to a fiber line filtrate pipeline of a pulp mill. The reactor, as well as the other parts of the system disclosed herein, can thus be installed to be an integrated unit of a pulp mill, and the fiber line filtrate can be conducted to the reactor.

In an embodiment the oxidative agent comprises, or is selected from, oxygen, ozone, air, or any combination thereof.

In an embodiment the oxidative agent is fed into the reactor and/or feedstock through a nozzle or an inlet port.

In an embodiment pH of the reaction mixture is adjusted to a value selected from the range 5-14, preferably from the range 7-13, more preferably from the range 10-12, or to a pH of about 11.

In an embodiment the reactor is operated at a temperature in the range 50-100° C., preferably in the range 80-95° C., more preferably in the range 85-95° C., most preferably at about 90° C.

In an embodiment the reactor is operated at a pressure selected from the range 0.5-10 bar (g), preferably from the range 1-7 bar (g), most preferably form the range 2-5 bar (g), or the pressure is about 4 bar (g).

In an embodiment the feedstock is, or comprises, filtrate from fiber line of a pulp mill. In an embodiment the feedstock comprises any filtrate comprising lignin. In another embodiment the feedstock comprises filtrate selected from brown stock washing filtrate, oxygen delignification filtrate, cleaned oxygen delignification filtrate, Ep-stage filtrate, Do-stage filtrate, Z-stage filtrate, or any combination thereof.

In an embodiment oxidized feedstock produced in the reactor is transferred back to a fiber line of a pulp mill after the oxidative treatment is finished. The oxidized filtrated can then be processed in the same way as filtrates are processed in the pulp mill.

In an embodiment oxidized feedstock is transferred to a stripping column, and the stripping gases produced in the stripping column are conducted to a condensation unit, where methanol is recovered. The recovered methanol can optionally be purified.

In an embodiment the reactor is operated in conditions wherein methanol is at least partially in vapor form, and wherein the reactor is directly in fluid connection to a liquefication unit or to a stripper off-gas (SOG) line. In an embodiment the SOG line collects vapors from strippers from the shell sides of any stripping system. The SOG is primarily methanol and TRS components. The SOGs can be then condensed to produce the liquid methanol. The SOGs are derived from different stripper units. These methanol collection units can thus be used to at least partially recover methanol produced by the present process, as well as methanol possibly present in the feedstock conducted to the reactor.

According to a second aspect there is provided a system comprising means for performing the process of the first aspect or any of its embodiment.

### BRIEF DESCRIPTION OF THE FIGURES

Some example embodiments will be described with reference to the accompanying figures, in which:

FIG. 1 schematically shows as an example embodiment certain parts of a system configured to carry out the present process.

### DETAILED DESCRIPTION

In the present description like reference signs denote like elements or steps.

In the present disclosure Adt refers to air dry ton.

In the present disclosure COD refers to chemical oxygen demand expressed as mg/l. The COD can be determined according to ISO 6060:1989, Water quality—Determination of the chemical oxygen demand.

As used herein, the term “comprising” includes the broader meanings of “including”, “containing”, and “comprehending”, as well as the narrower expressions “consisting of” and “consisting only of”.

In an embodiment the process steps are carried out in the sequence identified in any aspect, embodiment, or claim. In another embodiment any process step specified to be carried out to a product or an intermediate obtained in a preceding process step is carried out directly to said product or intermediate, i.e. without additional, optional or auxiliary processing steps that may chemically and/or physically alter the product or intermediate between said two consecutive steps.

In an embodiment the present process is an industrial process. In another embodiment the industrial process may exclude small scale methods such as laboratory scale methods that are not scaled up to volumes used in industry.

In an embodiment the present process is carried out without adding a catalyst to the reactor.

In an embodiment lignin present in the aqueous medium is at least partially dissolved or solubilized in the aqueous medium.

Lignin contains methoxyl groups that can be converted to methanol by the present process.

Lignin is either nonphenolic or phenolic, and approximately 40% of lignin is phenolic and 60% is non-phenolic. Phenolic lignin is reactive to mild oxidative conditions whereas non-phenolic lignin requires more oxidative conditions and more intense environment to produce methanol. With the operating conditions and the oxidative agent disclosed herein, methoxyl groups of phenolic and non-phenolic lignin available in the lignin structure can be converted to methanol.

In the present process the oxidation reactions can be executed in at least one oxidation reactor to convert the phenolic or/and non-phenolic methoxyl groups of lignin into methanol. In another embodiment more than one, such as two, three or four, reactors in a series are used.

In another embodiment the pressure and temperatures inside the reactor are selected such that methanol remains in liquid phase. The methanol can be dissolved in the feedstock.

In an embodiment pH of the reaction mixture is adjusted to a value selected from the range 5-14, preferably from the range 7-13, more preferably from the range 10-12, or about 11. The adjustment can be made with any alkali or acid. In an embodiment the pH adjustment is made with an alkali selected from sodium hydroxide, white liquor, and oxidized white liquor. The pH adjustment can be made initially at the beginning of the oxidative treatment, and/or during the oxidation reaction to keep the pH at or near the selected pH value.

In an embodiment the reactor is operated at a temperature in the range 50-100° C., preferably in the range 80-95° C., more preferably in the range 85-95° C., most preferably at about 90° C.

In an embodiment the reactor is operated such that the oxidation reaction is carried out up to 500 min, such as 10-500 min, 50-500 min, 100-500 min, 200-500 min. In an embodiment, when more than one reactor is used, the time refers to the total time the oxidative reaction conditions are maintained.

In another embodiment the reactor is operated such that the oxidation reaction is carried out up to 250 min, such as 50-200 min, 110-210 min, 100-200 min, or 90-190 min. In another embodiment the reaction is carried out for about 80, 100, 120, 180, or 200 min. In an embodiment, when more than one reactor is used, the time refers to the total time the oxidative reaction conditions are maintained.

In an embodiment non-condensable gases are removed by a non-condensable gas handling system.

In an example embodiment the filtrate comprises filtrate from a hardwood pulp mill. In an embodiment the filtrate has at least one component present in an amount shown in the analysis results Table 1, or within an error margin of 15% of any amount show in Table 1.

TABLE 1
Hardwood filtrate compositions.

| | | Post oxygen | Post oxygen |
|---|---|---|---|
| | Brown stock | washing filtrate | washing filtrate |
| | washing filtrate | (no cleaning) | (clean) |
| pH | 10-11 | 10-11 | 10-11 |
| COD, mg/l | 81600 | 19400 | 15400 |
| Lignin, mg/l | ~10000 | ~4000 | ~4000 |
| S, mg/l | 3 190 | 2 090 | 1 600 |

In an embodiment the reactor is operated in conditions comprising a temperature of 90° C., pH in the range 8-14, and a pressure of 8 bar, and by using oxygen as the oxidant for up to 200 min. In a preferable embodiment the pH is about 11.

In an embodiment the reactor is operated in conditions comprising a temperature of 90° C., pH in the range 8-14, and by using air as the oxidant for up to 200 min. In a preferable embodiment the pH is about 11.

In an embodiment the reactor is operated in conditions comprising a temperature of 90° C., pH in the range 8-14, and by using ozone as the oxidant for up to 30 min. In a preferable embodiment the pH is about 11.

The present process can produce about 2.5-5 kg methanol per Adt when oxygen delignification filtrate is used as the feedstock. For brown stock washing filtrate, the methanol production may be up to 8 kg/Adt due to a higher concentration of lignin in the filtrate. When using ozone as the oxidant, the methanol yield is increased even further and it can be 5 kg/Adt. In a preferable embodiment using brown stock filtrate, then even up to 8 kg/Adt of methanol can be produced. Ozone also allows using a shorter reaction time because ozone is more reactive than air or oxygen.

In an embodiment the oxidative agent is gaseous.

In an amount the amount of the oxidative agent used in the oxidative reactions is not more than 12 weight-% of the dry solids. In another embodiment the oxidative agent is used 1-12, 1-10, 3-12, 3-10, 5-12, 5-10, 7-12, or 7-10 weight-% of the dry solids.

In an embodiment the oxidative agent is fed into the reactor and/or feedstock through a nozzle or an inlet port. The nozzle or inlet port can be arranged inside the reactor, or on a wall, bottom plate, and/or top plate of the reactor.

In an embodiment the reactor is a stirred tank reactor. In another embodiment the reactor is a continuous stirred tank reactor, plug flow reactor, or a batch reactor.

In an embodiment the pressure in the reactor is selected from the range 0.5-10 bar (g), preferably from the range 1-7 bar (g), most preferably from the range 2-5 bar, or the pressure is about 4 bar.

In an embodiment the oxidized feedstock, such as an oxidized filtrate, is transferred back to the fiber line of the pulp mill after oxidative treatment with the present process. The fiber line may continue to an evaporation plant with a stripping column. The stripped gases can be taken to a liquefication unit, and to further purification of liquefied compounds. Because the oxidized feedstock fed into the fiber line contains an increased amount of methanol, the present process enhances production of methanol from lignin containing streams obtainable from pulp mills.

In an embodiment the oxidized feedstock is transferred to a stripping column, and stripping gases produced in the stripping column are conducted to a condensation unit where methanol is condensed for recovery, optionally followed by methanol purification. Methanol purification can be carried out for example by distillation.

Another advantage of the present process is, that existing methanol recovery means used in pulp mills, such as methanol evaporation, condensing and purification units, can be utilized to recover the methanol produced with the present process.

Alternatively or additionally, the methanol produced with the present process is recovered near the oxidation reactor. In this embodiment the reactor is operated such that at least part of the methanol is in a vapor form, and methanol can thus be recovered in a liquefication unit directly in fluid connection with the reactor. In one embodiment the oxidation reaction and the removal of methanol happen simultaneously. When the liquefication unit is directly in fluid connection with the reactor, at least part of the methanol present and formed in the oxidized feedstock is recovered before the oxidized feedstock is transferred back to the fiber line.

In a further alternative or additional embodiment, methanol is recovered from stripper off gases in fluid connection with the reactor. In an embodiment the off-gas stripper is directly in fluid connection with the reactor.

Alternatively or additionally, gases from the reactor are directed to an stripper off gases in fluid connection to at least one further source of gases produced in a pulp mill. In this configuration the methanol containing gases produced in the reactor can be processed with equipment present in pulp mills.

When oxidizing lignin with the present process, the lignin is partially degraded and the COD is reduced. The decreased COD can be used as an indication of methanol produced from the lignin present in the feedstock.

The oxidized filtrate can be returned to the fiber line to be mixed with the fiber line filtrate and for use in a washing process of the pulp mill. The oxidized filtrate can be returned to the fiber line without further processing or purification.

Because of the low sulphur content of fiber line filtrates, the present oxidation process can be executed with a minimal number of reactors in a series, such as in one or two reactors in a series. Because of the low amount of sulfuric compounds in the fiber line filtrates, the oxidative agent is no not significantly consumed by oxidation of the sulfuric compounds. Any reaction products resulting from the sulfur oxidations remain in the oxidized filtrate.

Because methanol boils at low temperature, most of the methanol can be extracted by using a simple stripping and/or distillation to separate volatile components/vapor from the liquid oxidized filtrate.

In an embodiment the present process is a continuous process, such as a process carried out in a continuous stirred tank reactor or a plug flow reactor.

In an embodiment the present process is a batch process.

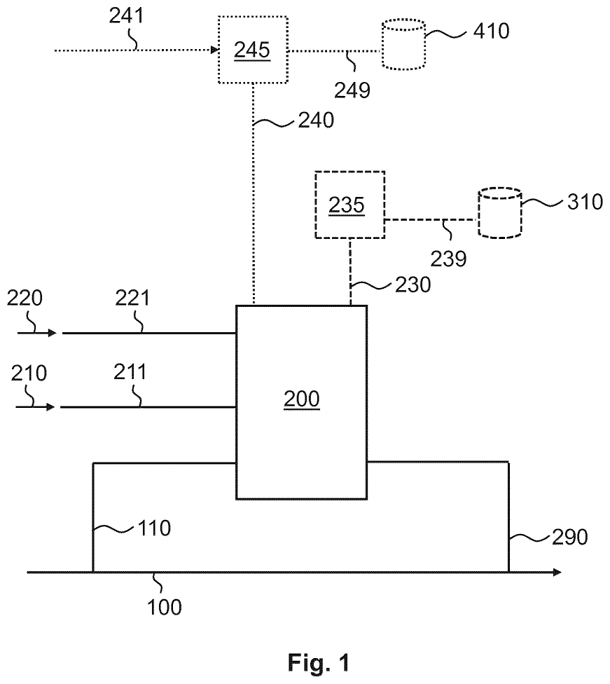

An example embodiment disclosing certain parts of a system configured to carry out the present process is illustrated in FIG. 1, in which the reactor 200 is connected to a fiber line 100 of a pulp mill via a reactor inlet line 110, which can be configured to feed fiber line filtrate into the reactor 200. A reactor outlet line 290 is connected to the fiber line 100 in a position downstream of the position in which the reactor inlet line 110 connects to the fiber line 100. To the reactor 200 is connected an oxidant feed inlet line 211, which can be configured to feed the oxidant into the reactor in a direction shown by the arrow 210. Alkali can be fed to the reactor through an alkali feed inlet line 221 in the direction shown by the arrow 220. In case acid or other chemical is fed into the reactor to adjust pH, said agents can be fed into the reactor through the inlet 221 or through other reactor inlets not shown in FIG. 1.

Methanol produced in the oxidation process is dissolved into the oxidized filtrate inside the reactor when the reactor is operated in conditions where methanol does not significantly evaporate. In such a case the methanol can be recovered from the oxidized feedstock, which is fed into the fiber line 100 by using equipment present in a pulp mill and which is used for removing methanol from the fiber line filtrate or from other methanol containing feedstocks.

In an alternative or additional embodiment, FIG. 1 shows a liquefication unit 235 and an SOG line 245 that can be used to remove methanol directly from the gas formed inside the reactor 200. In this configuration vapor is in connection to the stripper off gas line, where other vapors are collected as well in the pulp mill, and the off gases are then liquefied to produce liquid methanol, which can then be processed further.

In the embodiment shown in FIG. 1 these units are directly connected to the reactor 200. The reactor gas outlet 230 is in fluid connection to the liquefication unit 235 and it conducts gases and vapors, including methanol vapor, from inside the reactor to the liquefication unit 235. From the liquefication unit 235 the methanol condensed from vapor is conducted through a liquefication unit outlet 239 to a methanol storage 310. The methanol storage 310, or the liquefication unit 235, can also be configured to receive methanol from other methanol recovery units of the pulp mill, such as from a unit which recovers methanol from the fiber line filtrate 100.

In another alternative or additional embodiment, FIG. 1 shows a SOG line (stripper off gas line) 245 to which a reactor gas outlet 240 is in fluid connection to conduct gases and vapors, including methanol vapor, from inside the reactor 200 to the SOG line 245. From the SOG line 245 the methanol condensed from vapor is conducted through an outlet 249 to a methanol storage 410. The methanol storage 410 can be configured to receive methanol from other methanol recovery units of the pulp mill, such as from a unit which recovers methanol from the fiber line filtrate 100.

The SOG line collects preferably all the stripper off gases together, and the methanol produced by the present process can be combined to the existing line/collection, as shown by the inlet line 241.

The inlet and outlet lines that are configured to transfer material and are shown in FIG. 1, can be equipped with one or more valve and one or more pump to allow better control of the process, and to ensure efficient transfer of gaseous and liquid phases in different parts of the system. Sampling points can be arranged in pipes or vessels of the system to allow analysis of material and process parameters in the process.

Heating and/or cooling means can be arranged in the reactor to control the operating temperature of the reactor.

### EXAMPLE

Hard wood brown stock washing filtrate was treated in a 2-liter reactor (1 liter of filtrate was used). The filtrate was oxidized with oxygen for 3 hours in 2 bars and 90° C. The reaction was executed as a batch process and no more oxygen was fed into the reactor. The filtrate and oxygen were left to react for the 3 hours, and samples were taken every 30 mins. No catalyst was used, as the pH was high to begin with and it didn't decrease significantly.

The methanol concentration grew from 500 mg/l to 800 mg/l, which means an additional methanol production of 3.8 kg/Adt. The pH lowered from 13.1 to 12.9.

The results show that the present process was successful in producing methanol in mild process conditions from a feedstock containing lignin.

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

1. A process for producing methanol comprising:
providing, in a reactor, a feedstock comprising lignin in an aqueous medium;
feeding into the feedstock at least one oxidative agent to produce a reaction mixture in which methoxyl groups of the lignin are oxidized into methanol;
adjusting the pH of the reaction mixture to a value in a range of 5 to 14; and
operating the reactor at a temperature in a range of 50° C. to 100° C.

2. The process of claim 1, wherein the reactor is in fluid communication with a fiber line filtrate pipeline.

3. The process of claim 1, wherein the oxidative agent is selected from oxygen, ozone, air, or a mixture of oxygen, ozone and/or air.

4. The process of claim 1, wherein the oxidative agent is fed into the feedstock through a nozzle or an inlet port of the reactor.

5. The process of claim 1, wherein the adjusting of the pH is to a range of 7 to 13.

6. The process of claim 1, wherein the reactor is operated at a temperature in the range of 80° C. to 95° C. 80-95° C.

7. The process of claim 1, wherein the reactor is operated at a pressure in a range of 0.5 bar (g) to 10 bar (g).

8. The process of claim 1, wherein the feedstock is a filtrate from a fiber line of a pulp mill.

9. The process of claim 1, wherein the feedstock comprises filtrate selected from one or more of: brown stock washing filtrate, oxygen delignification filtrate, or cleaned oxygen delignification filtrate.

10. The process of claim 1, further comprising transferring oxidized feedstock from the reactor to a fiber line of a pulp mill.

11. The process of claim 1, further comprising transferring oxidized feedstock from the reactor to a stripping column,
generating stripping gases in the stripping column from the oxidized feedstock, and
transferring the stripping gases to a condensation unit to recover methanol from the stripping gases.

12. The process of claim 1, wherein the reactor is operated in conditions wherein methanol within the reactor is at least partially in vapor form, and the process further comprises transferring the methanol via a fluid connection to a liquefication unit or to a stripper off gas line.

13. (canceled)

14. A process including:
providing, in a reactor, a feedstock including lignin in an aqueous medium within the reactor;
feeding into the reactor and the feedstock an oxidative agent;
operating the reactor such that the feedstock in the aqueous median is at a pH in a range of 5 to 14;
operating the reactor such that the feedstock in the aqueous medium is in a temperature in a range of 50° C. to 100° C., and
producing methanol by reacting the oxidative agent with methoxyl groups of the lignin.

15. The process of claim 14, wherein the oxidative agent includes at least one of: oxygen, ozone or air.

16. The process of claim 14, wherein the pH is in a range of 7 to 13.

17. The process of claim 14, wherein the feedstock in the aqueous medium in the reactor is at a pressure in a range of 0.5 bar (g) to 10 bar (g).

18. The process of claim 14, wherein the feedstock comprises filtrate includes one or more of: brown stock washing filtrate, oxygen delignification filtrate, or cleaned oxygen delignification filtrate.

19. The process of claim 14, further comprising transferring oxidized feedstock from the reactor to a fiber line of a pulp mill.

20. The process of claim 14, further comprising transferring oxidized feedstock from the reactor to a stripping column,
generating stripping gases in the stripping column from the oxidized feedstock, and
transferring the stripping gases to a condensation unit to recover methanol from the stripping gases.
